(19) United States
(12) Patent Application Publication
(10) Pub. No.: US 2022/0098532 A1
(43) Pub. Date: Mar. 31, 2022

(54) LOW ALCOHOL BEER

(71) Applicant: Heineken Supply Chain B.V., Zoeterwoude (NL)

(72) Inventors: Eric Richard BROUWER, Zoeterwoude (NL); Paulien HOLLERING, Zoeterwoude (NL)

(21) Appl. No.: 17/548,098

(22) Filed: Dec. 10, 2021

Related U.S. Application Data
(63) Continuation of application No. PCT/EP2020/066492, filed on Jun. 15, 2020.

(30) Foreign Application Priority Data
Jun. 13, 2019 (EP) 19179912.1

(51) Int. Cl. C12C 12/04; C12C 7/04

(52) U.S. Cl. CPC C12C 12/04; C12C 7/04

(57) ABSTRACT

The invention provides a method of producing a low alcohol beer, comprising the steps of:
providing an alcoholic malt beverage;
subjecting the alcoholic malt beverage to a de-alcoholization step to produce a low alcohol malt beverage;
adding hops to the low alcohol malt beverage in an amount of at least 10 g/hl to produce at least 10 hl of a suspension;
keeping the suspension between 1 hour and 20 days at a temperature between −2 and 43° C. to produce a low alcohol beer; and optionally
subjecting said low alcohol beer to a filtration step.

This process results in a low alcohol beer which has a surprisingly good taste profile.

Also provided is a low alcohol beer that is obtained by the aforementioned method.

### CROSS-REFERENCE TO RELATED APPLICATIONS

The present application is a Continuation of International Patent Application No. PCT/EP2020/066492, filed Jun. 15, 2020, which claims priority to European Patent Application No. 19179912.1 filed Jun. 13, 2019; the entire contents of all of which are hereby incorporated by reference.

### TECHNICAL FIELD OF THE INVENTION

The present invention relates to a method of producing a low alcohol beer by de-alcoholisation of an alcoholic malt beverage, said method comprising addition of hops after the de-alcoholisation.

### BACKGROUND OF THE INVENTION

Beer is a universally popular beverage, consumed worldwide. Beer is commonly produced by a process that comprises the following basic steps:
mashing a mixture of grain and water to produce a mash;
separating the mash in wort and spent grain;
boiling the wort in the presence of hops, to produce a boiled wort;
fermenting the boiled wort with live yeast to produce a fermented wort;
subjecting the fermented wort to one or more further process steps (e.g. maturation and filtration) to produce beer; and
packaging the beer in a sealed container, e.g. a bottle, can or keg.

Hops are the flowers of the hop plant Humulus lupulus. They are used primarily as a bittering, flavouring, and stability agent. In addition to bitterness, hops impart floral, fruity, or citrus flavours and aromas. In recent years, the beer market has witnessed a significant increase in the consumption of non-alcoholic beer. This increase is triggered by concerns about health, and is expedited by innovations that have substantially improved the quality of non-alcoholic beers.

Low alcohol beers, including non-alcoholic beers, can be produced by a classical brewing process followed by alcohol removal (“de-alcoholization”) by techniques such as vacuum distillation, reverse osmosis, dialysis or evaporation.

De-alcoholization of beer is performed on regularly brewed beer, and is designed to remove ethanol, but as little as possible other flavor components. De-alcoholization may be achieved by, for instance, rectification, reverse osmosis or dialysis of regular beer. However, it is challenging to prevent flavor deprivation upon de-alcoholization of beer. Consequently, a drawback of de-alcoholized beer is a flat flavor, which may be corrected by addition of flavor (taste and odor) compounds. However, adding flavor is costly and requires at least one additional unit operation. Moreover, it is far from easy to obtain an agreeable taste by the addition of flavor after de-alcoholization due to the flavor of a beverage being the result of a complex and delicate balance between the constituents of the beverage, amongst others the quantity and type of various sugars, esters, aldehydes, as well as the quantity of alcohols, particularly ethanol. The presence of alcohol, particularly ethanol, suppresses some taste attributes, and enhances others.

U.S. Pat. No. 5,384,135 describes a process for the manufacture of an alcohol-free pale beer, comprising
the manufacture of a lager type alcoholic pale beer,
the dealcoholization of the said alcoholic pale beer by evaporation, under high vacuum,
at least one dilution with water, wherein the said water is deoxygenated and demineralized water having a total hardness of less than 1.8° dH.

Example 1 of the US patent describes a process in which dealcoholized beer concentrate (14-18% dry matter) is treated with charcoal, followed by:
dilution and addition of sugar solution,
addition of flavours,
addition of pre-isomerized and flavoured hop extract,
carbonation,
filtration, and
bottling.

Haslbeck et al. (Investigations into the Transfer Rate of Volatile Compounds in Dry Hopping Using an Octanol-Water Partition Coefficient Model, JOURNAL OF THE AMERICAN SOGE1YOF BREWING CHEMISTS, 2018, 76(3), 169-177) describes a study in which the effect of ethanol content on the transfer rates of volatile compounds in dry hopping was determined at different temperatures (1° C. and 20° C.). Nonalcoholic beer was used for the dry hopping trials. Hop pellets were added directly into beer bottles in dosages of 0.55-7.30 g/L, following which the bottles were resealed. After 7 days of dry hopping the samples were filtered, centrifuged and analysed.

Liguori et al. (Impact of Osmotic Distillation on the Sensory Properties and Quality of Low Alcohol Beer, Journal of Food Quality, vol. 2018, 2018, pages 1-11) describes a study in which the impact of osmotic distillation on the sensory properties of low alcohol beer was investigated. The article reports that hop (Humulus lupulus, cascade variety; 6.8% α-acids) extract and pectins solutions at a concentration of 2 mL each in 100 mL were added to 5 L of dealcoholized beer, followed by carbonation. The authors conclude that the addition of hop extract and pectin solution to low alcohol beer “better maintained hop and fruity-citrus notes during tasting, compared to the original beer”.

### SUMMARY OF THE INVENTION

The present invention provides a method of producing a low alcohol beer, preferably an alcohol-free beer, comprising the steps of:
providing an alcoholic malt beverage;
subjecting the alcoholic malt beverage to a de-alcoholization step to produce a low alcohol malt beverage;
adding hops to the low alcohol malt beverage in an amount of at least 10 g/hl to produce at least 10 hl of a suspension;
keeping the suspension between 1 hour and 20 days at a temperature between −2 and 43° C. to produce a low alcohol beer; and optionally
subjecting said low alcohol beer to a filtration step.

This process results in a low alcohol beer which has a surprisingly good taste profile.

### DETAILED DESCRIPTION OF THE INVENTION

A first aspect of the invention relates to a method of producing a low alcohol beer, preferably an alcohol-free beer, comprising the steps of:
providing an alcoholic malt beverage;
subjecting the alcoholic malt beverage to a de-alcoholization step to produce a low alcohol malt beverage;
adding hops to the low alcohol malt beverage in an amount of at least 10 g/hl to produce at least 10 hl of a suspension;
keeping the suspension between 1 hour and 20 days at a temperature between −2 and 43° C. to produce a low alcohol beer; and optionally
subjecting said low alcohol beer to a filtration step.

The term “a” or “an” as used herein is defined as “at least one” unless specified otherwise. When referring to a noun (e.g. a compound, an additive, etc.) in the singular, the plural is meant to be included. Thus, when referring to a specific moiety, e.g. “terpene”, this means “at least one” of a terpene, e.g. “at least one terpene, unless specified otherwise. The term “or” as used herein is to be understood as “and/or”.

When referring to a compound of which several isomers exist (e.g. a D and an L enantiomer), unless indicated otherwise, the compound includes all enantiomers, diastereomers and cis/trans isomers of that compound.

The term “alcohol” as used herein, unless indicated otherwise, is synonymous with “ethanol”.

The term “low alcohol” as used herein, unless indicated otherwise, means an alcohol content of between 0 and 1.0% alcohol by volume (ABV).

The term “alcohol free” as used herein means an alcohol content of between 0 and 0.1% ABV.

The present method is preferably employed in factory scale production of low alcohol beer. Accordingly, in a preferred embodiment, the low alcohol malt beverage is combined with hops to produce at least 10 hl of suspension, more preferably to product at least 50 hl of suspension and most preferably to produce 300-10,000 hl of suspension.

The alcoholic malt beverage which is subjected to the de-alcoholization step may be any beer. The alcoholic malt beverage may have an alcohol content of 1-12% ABV, preferably of between 2 and 8% ABV, more preferably of between 2.5 and 8% ABV, even more preferably between 3 and 7.5% ABV, yet more preferably between 4 and 7% ABV, and most preferably between 4.5 and 6% ABV.

In the present method preferably at least 50%, more preferably at least 70% and most preferably at least 80% of the alcohol present in the alcoholic malt beverage is removed in the de-alcoholization step.

In one embodiment the alcoholic malt beverage which is subjected to the de-alcoholization step is a lager. Alternatively, the alcoholic malt beverage which is subjected to the de-alcoholization step is an ale, more preferably an IPA (India Pale Ale).

Lager beer is typically produced using a bottom fermenting yeast such as S. eubayanus, C. carlsbergensis, or C. pasteurianum, whilst ale is typically produced using a top fermenting yeast such as S. cerevisiae.

The alcoholic malt beverage that is provided in the present method is preferably prepared by:
mashing a mixture of grain and water to produce a mash;
separating the mash in wort and spent grain;
boiling the wort;
fermenting the boiled wort with live yeast to produce a fermented wort;
subjecting the fermented wort to one or more further process steps (e.g. maturation and/or filtration) to produce beer; and
packaging the beer in a sealed container, e.g. a bottle, can or keg.

The alcoholic malt beverage that is provided in the present method may suitably be produced by adding hops and/or hops extract to wort, preferably before or during wort boiling. In this preferred embodiment of the method, hops are introduced twice, i.e. in the form of hops or hop extract during the preparation of the wort and in the form of hops after the de-alcoholization step.

De-alcoholization can be done using techniques known in the art, such as reverse osmosis, dialysis or evaporation (see for general methodology Mangindaan et al., Trends in Food Science and Technology 71 (2018), 36-45; and Brányik et al, J. Food Eng. 108 (2012), 493-506).

In a preferred embodiment de-alcoholization is done using distillation, more preferably vacuum distillation. Vacuum distillation is a technique which is well known in the art.

The skilled person is capable of determining suitable conditions for distillation based on common general knowledge and the information disclosed herein.

In an embodiment, distillation is performed at a temperature of between 10-100° C., more preferably 20-65° C., more preferably 30-50° C., even more preferably 40-46° C.

Distillation may be performed at reduced pressure, such as at a pressure of 0.01-500 mbar, preferably 1-200 mbar, more preferably 50-150 mbar, and even more preferably 80-110 mbar.

Vacuum distillation may comprise one or more of the steps of:
pre-heating of the alcoholic malt beverage, for example in a heat exchanger
degassing of the alcoholic malt beverage, for example in a vacuum degasser
separation of ethanol from the alcoholic malt beverage, in one or more vacuum columns, for example a packed-bed column
cooling and optional carbonation.

The skilled person is capable of selecting suitable equipment to perform the distillation. The distillation may, for example, be performed in a commercial rectification column. The skilled person is capable of selecting a suitable setup on the basis of common general knowledge, such as described in “Chemical Engineering Design”, by R. K. Sinnott (Volume 6, fourth edition, 2005).

Any type of hops can be added to the low alcohol malt beverage, depending on the desired type of low alcohol beer. The hops may be added for example in the form of hop bells and/or hops pellets in a typical amounts of between 10 and 1200 g/hl. The skilled person knows how to select the type and amount of hops, based on the common general knowledge and/or the instructions provided by the hops supplier. For example, to make a hoppy beer such as an IPA, typically more hops are added, whereas for the lager type of beer, less hops are added. Preferably, hops are added to the low alcohol malt beverage in an amount of 10-1200 g/hl, more preferably in an amount of 20-500 g/hl hops to produce a suspension.

Adding hops to the low alcohol malt beverage results in a suspension. This suspension is kept between 1 hour to 20 days before being filtered, so that the hops can release flavor and aroma, resulting in a low alcohol beer.

In a preferred embodiment, after combination with the hops, the suspension is kept between 2 hours and 15 days, more preferably between 3 hours and 10 days, between 4 hours and 6 days, between 6 hours and 4 days, and most preferably between 12 hours and 3 days to produce the low alcohol beer.

After a certain time, most of the flavor and aroma will have been released from the hops and maintaining the suspension any longer will not release much more flavor.

The lower time limit is governed by the amount of aroma and flavor compounds that needs to be released from the hops.

In a preferred embodiment, the aforementioned suspension is kept at a temperature that does not exceed 35° C. before filtration. The temperature at which the suspension is kept preferably does not exceed 30° C., more preferably does not exceed 25° C., 20° C., 15° C., 12° C., 10° C., 8° C., 6° C., and most preferably does not exceed 4° C. The time period during which the suspension is kept is typically shorter at higher temperatures and longer at lower temperatures.

The lower temperature limit is determined by the freezing temperature. Maintaining the suspension at lower temperatures may advantageously prevent microbial contamination. Maintaining the suspension at higher temperatures results in more rapid release of flavor and aroma.

The skilled person can easily establish a suitable combination of time and temperature by drawing samples and evaluating the taste of suspension over time, to judge whether a good quality low alcohol beer has been formed.

Prior to adding hops, trub may be removed from the alcoholic malt beverage or the low alcohol malt beverage in a wort clarifier, such as a whirlpool. Preferably, trub is removed before the de-alcoholization of the alcoholic malt beverage.

The present method results in a low alcohol beer which has surprisingly good taste profile without the need of adding flavors, or by adding less flavors than taught in the art. Thus, in a preferred embodiment the method comprises adding 0-20 ppm ethyl acetate and/or 0-5 ppm isoamyl acetate, relative to the volume of the low alcohol beer.

Preferably, the method comprises adding 0-15 ppm ethyl acetate, 0-10 ppm ethyl acetate, 0-7 ppm ethyl acetate, 0-5 ppm ethyl acetate, 0-4 ppm ethyl acetate, 0-3 ppm ethyl acetate, 0-2 ppm ethyl acetate, 0-1 ppm ethyl acetate, and most preferably no ethyl acetate is added.

The method preferably comprises adding 0-5 ppm isoamyl acetate, 0-4 ppm isoamyl acetate, 0-3 ppm isoamyl acetate, 0-2 ppm isoamyl acetate, 0-1 ppm isoamyl acetate, and most preferably no isoamyl acetate is added.

In one advantageous embodiment no ethyl esters are added in the present method. In another advantageous embodiment no acetate esters are added in the method. In a particularly preferred embodiment of the present method no ethyl esters and no acetate esters are added.

According to a particularly preferred embodiment no flavors are added in the present method.

In an advantageous embodiment of the present method, the method comprises adding sugar, for example adding a sugar syrup. Suitable sugars include sucrose and glucose. Adding sugar may add sweetness and contribute to the mouthfeel. Sugar is preferably added in the method as late as possible, preferably after de-alcoholization, more preferably after filtration, to prevent ethanol formation.

In a particularly preferred embodiment of the present method the low alcohol beer that is obtained by the method described herein before is combined with another low alcohol beer that has been produced by a different route so as to produce a low alcohol beer having an exceptionally good flavor. More particularly, in accordance with this embodiment of the present method, the method produces a first low alcohol beer and further comprises:
a. separately preparing a second low alcohol beer by contacting a heat-treated wort with yeast under conditions of restricted fermentation to produce a second low alcohol beer; and
b. combining at least part of the first low alcohol beer with at least part of the second low alcohol beer.

At the end of the present method the low alcohol containing beer can be introduced into a sealed container. Examples of suitable containers include bottles, cans, kegs and tanks.

method as described above.

The present method preferably yields a low alcohol beer that is rich in xanthohumol, and relatively low in isoxanthohumol. Accordingly, the low alcohol beer produced by the present method preferably comprises xanthohumol and isoxanthohumol in a ratio (w/w) of at least 0.2, preferably at least 0.5, more preferably at least 1, even more preferably at least 2, at least 3, at least 4, and most preferably at least 5. The inventors have found that such a low alcohol beer offers one or more advantages compared to low alcohol containing beers having a xanthohumol:isoxanthohumol ratio (w/w) of more than 0.2, including:
increased foam stability;
reduced susceptibility to aging, e.g. less susceptibility to staling;
less formation of Strecker aldehydes, especially less formation of methylpropanal, 2-methylbutanal, 3-methylbutanal, phenylacetaldehyde and/or methional;
less formation of furfural

The amount of xanthohumol in the low alcohol beer preferably ranges between 100 and 5000 μg/L, between 200 and 2000 μg/L, between 250 and 1500 μg/L, between 300 and 1200 μg/L, and most preferably between 500 and 1000 μg/L.

The amount of isoxanthohumol in the low alcohol beer preferably ranges between 5 and 500 μg/L, between 10 and 450 μg/L, between 20 and 400 μg/L, between 30 and 350 μg/L, and most preferably between 40 and 300 μg/L.

The invention is illustrated by means of the following non-limiting examples.

### EXAMPLES

### Methods

Analysis of Xanthohumol and Isoxanthohumol

Concentrations of xanthohumol and isoxanthohumol were determined by UPLC-MS using the procedure described below.

Standards (xanthohumol, CAS 6754-58-1; isoxanthohumol, CAS 70872-29-6) were bought from Cayman chemical company and Sigma-Aldrich respectively. The concentrations of each substance were determined on the basis of calibration curves for each individual compound, spiked in a commercial alcohol free beer. The calibration curves for the two substances were determined using the detector conditions described below. The equipment, materials and conditions described below were used to determine the concentrations of the aforementioned hop derived substances.

Equipment:
UPLC system: Acquity UPLC (Waters) equipped with a BEH C 18 column (1.7 μm, 2.1 mm×150 mm; Waters product number: 186002353)
Detector: Synapt G2 ToF mass spectrometer (Waters)
Milli-Q® Ultrapure Water
Acetonitrile>99.9% (JT-Baker)
Formic acid>98% (Sigma-Aldrich)

UPLC Instrument Conditions:
Column temperature: 50° C.
Injection volume: 10 μl
Sample temperature: 10° C.
Run time: 30 min
Eluent A=999 mL Ultrapure water+4-1 mL formic acid
Eluent B=999 mL acetonitrile+1 mL formic acid

Gradient:
T 0 min: 95% A/5% B
T 25 min: 5% A/95% B
T 27-30 min 95% A/5% B

Detector Conditions:
Analytical method is set-up in negative scan mode (ES−)
MS settings:
ToF MS range: 100-1500 amu
Capillary Voltage; 2.5 kV
Cone Voltage: 40V
Source temperature 120° C.
Desolvation temperature: 350° C.

Both xanthohumol and isoxanthohumol were determined by extracting m/z 353 (M—H)— from the data set. Samples were degassed prior to analysis.

### Example 1

Five hundred liters of a lager, containing 5% alcohol ABV, were produced by mashing a mixture of grain and water to produce a mash; separating the mash in wort and spent grain; boiling the wort in the presence of hops to produce a boiled wort; fermenting the boiled wort with live yeast to produce a fermented wort; and subjecting the fermented wort to maturation and filtration to produce said lager.

Subsequently, the beer was de-alcoholized by vacuum distillation (Schmidt-Bretten, Bretten, Germany—settings: feed, 5 hL/hr; steam mass flow rate, 100 kg/h; outlet pressure, 3.5 bar; vacuum setting, 90 mbar; outlet temperature, 3° C.). Next, the de-alcoholized beer, having an alcohol content of less than 0.1% ABV, was carbonized to approximately 5 g/L CO₂.

The thus de-alcoholized, carbonized beer having an extract of 7.6° was fed to a container and cooled to 2° C. Aroma hop pellets (Yakima Chief, P90) were subsequently added to the beer at a dosage of 350 g hop/hL. The beer was recirculated for 1 day at 2° C. using a pump which simultaneously pulvarized the hop pellets. Next, the hopped, de-alcoholized, carbonized beer was stored for 5 days at 2° C. allowing the hop particles to settle.

Next, the beer was filtrated over a membrane, and diluted with water to 4.9°. Finally, sucrose (1.5 kg/hL of 67 brix) was added to arrive at a final sugar concentration of 5.9°. No flavors were added.

The resulting low alcohol containing beer was analyzed for xanthohumol and isoxanthohumol. The results are shown in Table 1.

TABLE 1
Concentration of xanthohumol and isoxanthohumol

| | |
|---|---|
| Xanthohumol | 0.7 ppm |
| Isoxanthohumol | 0.2 ppm |

The beer was subjected to sensory analysis by an external, professional expert panel consisting of 17 persons. The beer was presented in black, non-transparent glass containers. After tasting, the beer was evaluated by plenary discussion. The panel members concluded that the beer combined a pleasant hoppy taste with the delicate, fruity, fresh taste of citrus fruit and tropical fruit. Also, the bitterness of the beer was judged to be pleasant. Overall, the panel considered the alcohol free beer to have a very pleasant taste.

1. A method of producing a low alcohol beer, comprising:
(a) subjecting an alcoholic malt beverage to de-alcoholization to produce a low alcohol malt beverage;
(b) adding hops to the low alcohol malt beverage in an amount of at least 10 g/hl to produce at least 10 hl of a suspension;
(c) keeping the suspension between 1 hour and 20 days at a temperature between −2 and 43° C. to produce a low alcohol beer; and
(d) subjecting the low alcohol beer to filtration.

2. The method according to claim 1, further comprising adding, to the low alcohol malt beverage, to the low alcohol beer or to the filtered low alcohol beer, 0-20 ppm ethyl acetate and/or 0-5 ppm isoamyl acetate, the added amount of ethyl acetate and/or isoamyl acetate being calculated relative to the volume of the low alcohol beer.

3. The method according to claim 1, which does not comprise adding acetate esters.

4. The method according to claim 1, which does not comprise adding ethyl esters.

5. The method according to claim 1, which does not comprise adding flavours.

6. The method according to claim 1, wherein the low alcohol malt beverage is filtered after the suspension has been kept between 1 hour and 20 days at a temperature between −2 and 43° C.

7. The method according to claim 1, wherein the low alcohol beer is a low alcohol lager.

8. The method according to claim 1, wherein the low alcohol beer is a low alcohol ale.

9. The method according to claim 1, wherein the alcoholic malt beverage has an alcohol content of between 1 and 12% ABV.

10. The method according to claim 9, wherein at least 50% of the alcohol present in the alcoholic malt beverage is removed during de-alcoholization.

11. The method according to claim 1, wherein the de-alcoholization comprises vacuum distillation.

12. The method according to claim 1, further comprising:
(i) mashing a mixture of grain and water to produce a mash;
(ii) separating the mash in wort and spent grain;
(iii) boiling the wort;
(iv) fermenting the boiled wort with live yeast to produce a fermented wort;
(v) subjecting the fermented wort to one or more further process steps; and
(vi) packaging the beer in a sealed container,
wherein hops and/or hops extract is added to wort before or during the wort boiling.

13. The method according to claim 1, comprising subjecting the low alcohol containing beer to filtration and adding sugar to the low alcohol beer after filtration.

14. The method according to claim 1, wherein the method produces a first low alcohol beer and further comprises:
(a) separately preparing a second low alcohol beer by contacting a heat-treated wort with yeast under conditions of restricted fermentation to produce a second low alcohol beer; and
(b) combining at least part of the first low alcohol beer with at least part of the second low alcohol beer.

15. The method according to claim 1, wherein the low alcohol beer has an alcohol content of between 0 and 1.0% ABV.

16. The method according to claim 1, wherein the low alcohol beer comprises xanthohumol and isoxanthohumol in a ratio (w/w) of at least 0.2.

17. The method according to claim 1, wherein the low alcohol beer comprises xanthohumol and isoxanthohumol in a ratio (w/w) of at least 0.5.

18. The method according to claim 1, wherein the low alcohol beer comprises xanthohumol and isoxanthohumol in a ratio (w/w) of at least 1.

19. The method according to claim 16, wherein the concentration of xanthohumol is between 100 and 5000 μg/L.

20. The method according to claim 19, wherein the concentration of isoxanthohumol is between 5 and 500 μg/L.
